(19) United States
(12) Patent Application Publication
(10) Pub. No.: US 2020/0093821 A1
(43) Pub. Date: Mar. 26, 2020

(54) Pharmaceutical Compositions of a Benzothiophene Compound

(71) Applicant: Hexal AG, Holzkirchen (DE)

(72) Inventors: Andreas Krekeler, Holzkirchen (DE); Dimitri Neumann, Holzkirchen (DE); Helmut Lasinger, Holzkirchen (DE); Herbert Silberberger, Kundl (AT); Ludwig Englmeier, Holzkirchen (DE)

(73) Assignee: Hexal AG, Holzkirchen (DE)

(21) Appl. No.: 16/325,520

(22) PCT Filed: Aug. 11, 2017
(86) PCT No.: PCT/EP2017/070431

§ 371 Date: Feb. 14, 2019

(30) Foreign Application Priority Data
Aug. 16, 2016 (EP) 16184402.2

(51) Int. Cl. A61K 31/496; A61K 9/00; A61J 1/14

(52) U.S. Cl. CPC A61K 31/496; A61J 1/1468; A61K 9/0019

(57) ABSTRACT

The present invention discloses possibilities to make use of a substance or a material arranged to block, absorb and/or reflect UV exposure of a certain wavelength region to reduce or prevent UV-induced dimerization and optionally further UV-induced brexpiprazole impurities of brexpiprazole in a brexpiprazole dihydrate comprising pharmaceutical composition.

### FIELD OF THE INVENTION

The present invention relates to a pharmaceutical composition comprising brexpiprazole and to possibilities to improve such a pharmaceutical composition in terms of its purity characteristics.

### BACKGROUND OF THE INVENTION

7-[4-[4-(1-Benzothiophen-4-yl)piperazin-1-yl]butoxy]quinolin-2(1H)-one (brexpiprazole; compound I) is an antidepressant and antipsychotic drug marketed under the brand Rexulti® for the oral treatment of schizophrenia and as an adjunctive treatment to antidepressants in major depressive disorder. REXULTI tablets are intended for oral administration and available in 0.25 mg, 0.5 mg, 1 mg, 2 mg, 3 mg and 4 mg strengths. The product was approved in the U.S. in 2015 for the aforementioned indications and is currently in phase III trials for the treatment of agitation associated with Alzheimer's disease and the treatment of PTSD (post-traumatic stress disorder). Rexulti® comprises crystalline brexpiprazole anhydrate.

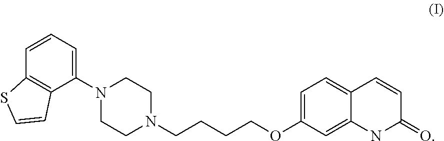 [SMILES: O=c1ccc2ccc(OCCCCN3CCN(c4cccc5sccc45)CC3)cc2[nH]1]

Brexpiprazole is an atypical antipsychotic and shows partial agonist activity at serotonin 5-HT1A and dopamine D2 receptors, and antagonist activity at serotonin 5-HT2A receptors.

WO 2006/112464 A1 discloses piperazine-substituted benzothiophenes, as a class of compounds comprising brexpiprazole, for the treatment of mental disorders such as schizophrenia and other central nervous system disorders.

WO 2012/137971 A1 relates to combinations comprising brexpiprazole and a second drug for use in the treatment of a central nervous system disorder.

WO 2013054872=EP 2 767 285 A1 discloses coated and uncoated tablets containing brexpiprazole. Experiments were carried out that give information on the photostability of brexpiprazole in tablets that are coated with a coating comprising specific photo-protecting excipients when compared to the respective uncoated tablets.

Pharmaceutical compositions of brexpiprazole have been launched and seem to satisfy regulatory needs, however they might still not yet satisfy all relevant needs. Accordingly there remains the problem to provide a dosage form of brexpiprazole with improved performance and use attributes, including the possibility of providing superior purity characteristics. In particular, there is a need for pharmaceutical compositions different from tablets, e.g. injectable compositions, that exhibit satisfying properties also and particularly with regard to improved purity characteristics.

### SUMMARY OF THE INVENTION

The present inventors have identified a previously unknown impurity of brexpiprazole, which appeared occasionally and to variable degree in the pharmaceutical compositions that the inventors were preparing. The present inventors have then studied the nature of the impurity and the underlying reasons for its variable appearance and have thus discovered that this UV-induced impurity generation, in particular the generation of a UV-induced compound identified as brexpiprazole dimer, can be significantly reduced or entirely prevented if the brexpiprazole present in a pharmaceutical composition is effectively prevented from exposure to UV-light having a specific wavelength region.

In prior art (e.g. in WO 2013054872=EP 2 767 285 A1) it is shown that tablets that contain brexpiprazole exhibit comparably low photoinstability. For instance, when comparing the level of light-induced impurities in tablets that are coated with a coating containing an excipient that provides protection against light on the one hand with the level of light-induced impurities in corresponding tablets that do not have this coating on the other, it was shown that the resulting impurities in the uncoated tablets, that is, in tablets that were not protected against light, was detectable, but comparatively low.

Thus, brexpiprazole—at least in the form as it is present in said tablets—is comparatively stable upon exposure to light.

Distinct from general impurity considerations, and in particular distinct from the finding in the prior art relating to brexpiprazole tablets, it was found in the present invention that brexpiprazole in its dihydrate form, also referred to herein as brexpiprazole dihydrate, is specifically prone to UV-induced dimerization. In this connection it was also found that crystalline brexpiprazole dihydrate is significantly more sensitive to UV-induced dimerization when compared to crystalline brexpiprazole anhydrate. It has further been found in the present invention that the product resulting from the exposure of brexpiprazole dihydrate to UV-radiation is a brexpiprazole dimer exhibiting the chemical structure of Formula I:

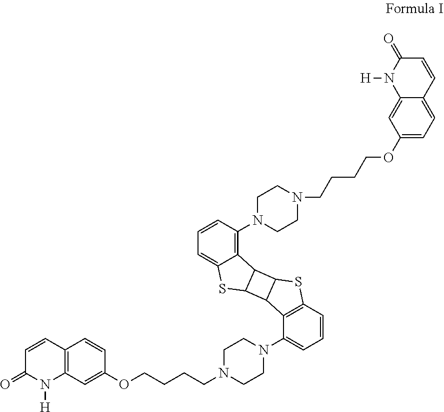 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

As already mentioned, brexpiprazole is currently marketed in form of a tablet as Rexulti®. In this tablet, brexpiprazole is present as brexpiprazole anhydrate, which is comparatively stable upon UV-irradiation.

However, although there are pharmaceutical compositions on the market that comprise brexpiprazole and seem to fulfil regulatory requirements, such as the oral dosage form Rexulti®, there is a need for further pharmaceutical compositions, in particular injectable formulations, that comprise brexpiprazole dihydrate and additionally fulfil regulatory requirements, in particular with regard to their impurity content.

Based on the surprising finding that brexpiprazole dihydrate is highly susceptible to UV-induced dimerization, and that upon exposure of brexpiprazole dihydrate to UV-light a brexpiprazole dimer that is characterized by anyone of the following characteristics (i) to (iv), respectively alone or in combination, preferably characteristic (iv) and, optionally, additionally any one of characteristics (i) to (iii):

(i) UV-chromatographic RRT of 0.92±0.02

(ii) LC-MS molecular weight [M+H]+=867±1,

(iii) chemical formula C₅₀H₅₄N₆O₄S₂,

(iv) chemical structure of Formula I:

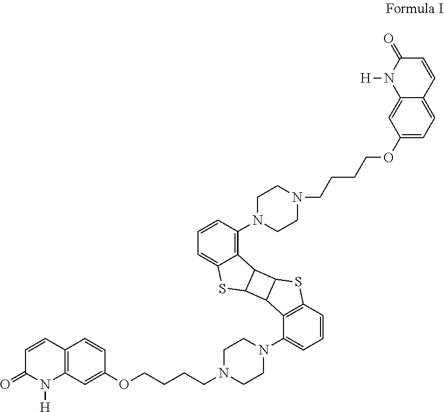 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

is generated, the present invention provides an improved pharmaceutical composition, in particular an injectable pharmaceutical preparation/composition, comprising brexpiprazole dihydrate and at the same time fulfilling regulatory requirements in particular with regard to impurities. The present invention further provides an improved method for preparing such a composition.

Further based on the above finding it was found that the UV-induced generation of brexpiprazole dimer, in particular a brexpiprazole dimer exhibiting a chemical structure of Formula I, can be prevented by using a means capable of preventing exposure of brexpiprazole dihydrate to UV-light having a wavelength of up to 450 nm. The means can be in the form of a substance capable to block, absorb or reflect the UV-light or it can be in the form of a material which is arranged in association with the pharmaceutical composition comprising brexpiprazole dihydrate, preferably such that UV irradiation is blocked, absorbed and/or reflected. The UV light to be kept from interacting with the brexpiprazole dihydrate is UV light comprising wavelengths of up to 450 nm, particularly the UVB wavelength region (280-315 nm) and/or the UVA wavelength region (315-450 nm).

As said before, it has been surprisingly found in the present invention that exposure of brexpiprazole dihydrate to UV light having a wave length as defined elsewhere herein results in the formation of a brexpiprazole dimer that is characterized by anyone of the following characteristics (i) to (iv), respectively alone or in combination, preferably characteristic (iv) and, optionally, additionally any one of characteristics (i) to (iii):

(i) UV-chromatographic RRT of 0.92±0.02

(ii) LC-MS molecular weight [M+H]+=867±1,

(iii) chemical formula C₅₀H₅₄N₆O₄S₂,

(iv) chemical structure of Formula I:

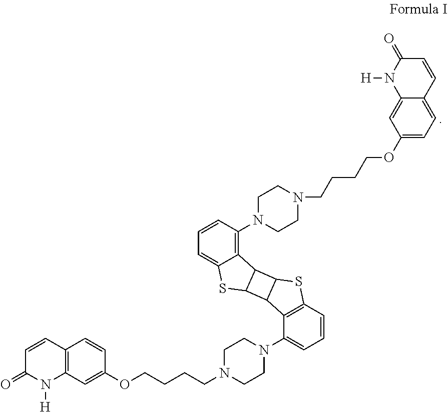 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

Based thereon, the present invention further provides pharmaceutical compositions, such as injectable compositions, comprising brexpiprazole dihydrate. By using a means that is capable of preventing exposure of brexpiprazole dihydrate to UV light exhibiting a wave length as defined herein, the level of UV-induced brexpiprazole dimers can be reduced or their generation can be prevented in a pharmaceutical composition compared to pharmaceutical compositions comprising brexpiprazol dihydrate that have been exposed to UV-light, as would happen during regular preparation, handling and/or storage of such a pharmaceutical composition.

Thus, the present invention also relates to a pharmaceutical composition, preferably an injectable pharmaceutical composition, comprising brexpiprazole dihydrate and a substance present in an amount sufficient to reduce or prevent UV-induced dimerization of brexpiprazole, preferably UV-induced dimerization of brexpiprazole resulting in a brexpiprazole dimer as disclosed elsewhere herein.

The present invention also relates to a package including one or more pharmaceutical compositions, preferably an injectable pharmaceutical composition, comprising brexpiprazole dihydrate, wherein said package comprises a packaging material, wherein said packaging material is capable of blocking, absorbing, and/or reflecting UV exposure up to a wavelength of 450 nm.

As disclosed herein, brexpiprazole dimers in general, and in particular the brexpiprazole dimers exhibiting anyone of characteristics (i) to (iv), preferably characteristic (iv) and, optionally, additionally any one of characteristics (i) to (iii), preferably characteristic (i), as disclosed elsewhere herein, represent impurities that in the context of the present invention are desired to be avoided.

Based thereon, it is possible to assess whether a batch of brexpiprazole dihydrate is suitable for the preparation of a pharmaceutical composition comprising brexpiprazole dihydrate.

Thus, the present invention also relates to a method of evaluating the suitability of a batch of brexpiprazole dihydrate for the preparation of a pharmaceutical composition, preferably an injectable composition, as well as to the use of a brexpiprazole dimer exhibiting anyone of characteristics (i) to (iv), preferably characteristic (iv) and, optionally, additionally anyone of characteristics (i) to (iii), preferably (i), for evaluating the suitability of a batch of brexpiprazole dihydrate for the preparation of a pharmaceutical composition.

Accordingly, the present invention provides the following aspects, subject-matters and preferred embodiments which, respectively taken alone or in combination, contribute to providing improved technical effects and to solving the afore-mentioned object of the invention:

1. Use of a means capable of preventing exposure of brexpiprazole dihydrate to UV light comprising wavelength of up to 450 nm for the prevention or reduction of UV-induced brexpiprazole dimerization.

In a preferred embodiment, essentially all of the brexpiprazole is present as brexpiprazole dihydrate.

2. Use of a means capable of preventing exposure of brexpiprazole dihydrate to UV light comprising wavelength of up to 450 nm for the prevention or reduction of UV-induced brexpiprazole impurities.

In a preferred embodiment, essentially all of the brexpiprazole is present as brexpiprazole dihydrate.

3. The use according to item 1 and/or 2, wherein the means is a substance capable to block, absorb and/or reflect UV exposure in a wavelength region up to 450 nm.

4. The use according to anyone of the preceding items, wherein the means is a material arranged to block, absorb and/or reflect UV exposure in a wavelength region up to 450 nm.

5. The use according to anyone of the preceding items, wherein the substance or substance arrangement or material is selected from the group consisting of:

(i) use of a substance or arrangement of a pharmaceutical composition, such as a tablet structure or an injectable pharmaceutical composition structure, per se capable of reducing or preventing UV-induced dimerization of brexpiprazole,

(ii) a substance present in an amount sufficient to reduce or prevent UV-induced dimerization of brexpiprazole, and/or

(iii) a packaging or container material at least partially, preferably entirely enclosing the brexpiprazole dihydrate comprising pharmaceutical composition, and capable of blocking, absorbing and/or reflecting UV exposure up to a wavelength of 450 nm.

6. The use according to item 2 or 5, wherein said substance or material is arranged to block, absorb and/or reflect UV irradiation in the UVC wavelength region (200-280 nm), the UVB wavelength region (280-315 nm) and/or the UVA wavelength region (315-450 nm), preferably in the UVB wavelength region and/or the UVA wavelength region.

7. The use according to item 5 or 6, wherein in case of (ii) the substance is present in an amount of at least 1 wt. %, preferably at least 2 wt. % and more preferably at least 3 wt. % relative to the weight of the compartment of the pharmaceutical composition comprising brexpiprazole, which optionally is a tablet core or an injectable pharmaceutical composition; and/or

the substance is present in a weight ratio relative to the weight of brexpiprazole present in the pharmaceutical composition of 0.2:1 or higher, preferably 0.2-3:1, more preferably 0.2-2:1, even more preferably 0.3-1:1.

8. The use according to anyone of the preceding items, which leads to a reduced or prevented amount of brexpiprazole dimer that is characterized by anyone of the following characteristics (i) to (iv), respectively alone or in combination; preferably characteristic (iv) and, optionally, additionally any one of characteristics (i) to (iii):

(i) UV-chromatographic RRT of 0.92±0.02

(ii) LC-MS molecular weight [M+H]+=867±1,

(iii) chemical formula C₅₀H₅₄N₆O₄S₂,

(iv) chemical structure of Formula I:

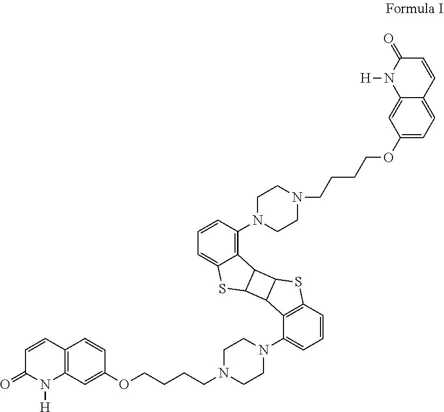 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

For (i) the following HPLC conditions can be used:

| Parameter | Dionex 3000 | | |
|---|---|---|---|
| Column | YMC Pack Pro C18 - 3.0 μm - 100 × 4.6 mm | | |
| Temperature Columnt | 45° C. | | |
| Compartmen | | | |
| Solvent A | 0.1% acetic acid pH 2.3/ACN - 85:15 ⇒ Gradient | | |
| Solvent B | 0.1% acetic acid pH 2.3/ACN - 25:75 ⇒ Gradient | | |
| | Time [min] | % A | % B |
| *) Gradient | 0 | 100 | 0 |
| | 25 | 69 | 40 |
| | 30 | 0 | 100 |
| | 30.1 | 100 | 0 |
| | 37 | 100 | 0 |

| | |
|---|---|
| Flow | 0.8 ml/min—p ≈107 bar |
| Wavelength | 254 nm |
| Injection Volume | 10 μl |
| Temperature Auto | RMT |
| Sampler | |
| Sample Concentration | 0.5 mg/ml |
| Run Time | 37 min - RT (Brexpiprazole) ≈ 13 min |

For (ii) the following LC-MS measurement conditions can be used:

| MS Tune Parameters | System: HCT ultra |
|---|---|
| Ionization Mode | ESI |
| Polarity | positive |
| Mass Range | 200-2000 |
| MSⁿ-Mode | MS/MS |
| Tune Mass [amu] | 450 |
| Nebulizer Pressure [psi] | 60 |
| Drying Gas Flow [L/min] | 11 |
| Drying Gas Temperature [° C.] | 365 |

9. A tablet comprising at least a tablet core, wherein the tablet core comprises brexpiprazole dihydrate in combination with a substance which is selected from the group consisting of inorganic pigments and organic pigments, solvent soluble dyes, water soluble dyes and organic lakes and which blocks, absorbs and/or reflects UV irradiation.

In a preferred embodiment, essentially all of the brexpiprazole is present as brexpiprazole dihyd rate.

10. The tablet according to anyone of item 9, wherein said substance blocks, absorbs and/or reflects UV irradiation up to a wavelength of 450 nm, preferably in the UVC wavelength region (200-280 nm), the UVB wavelength region (280-315 nm) and/or the UVA wavelength region (315-450 nm), more preferably in the UVB wavelength region and/or the UVA wavelength region.

11. The tablet according to item 9 or 10, wherein said inorganic pigments are selected from titanium dioxide, zinc oxide, red iron oxide, yellow iron oxide, black iron oxide, said organic pigments are selected from D & C Red 30, D & C Red 34 and D & C Red 36; said solvent soluble dyes are selected from Green 6, D & C Red 17, D & C Violet 2, D & C Yellow 7, D & C Yellow 11, D & C Red 21, D & C Red 27, and D & C Orange 5, said water soluble dyes are selected from D & C Green 5, FD & C Green 3 (Fast Green), FD & C Yellow 5 (Tartrazine), FD & C Yellow 6 (Sunset Yellow), Quinoline Yellow, D & C Yellow 10, FD & C Red 3 (Erythrosine), D & C Red 22, FD & C Blue 1 (Brilliant Blue), FD & C Blue 2 (Indigotine), FD & C Blue 3, Ponceau 4R, Carmoisine, Naphthol Blue lack, Amaranth, Patent Blue V, Black PN, Orange G, D & C Green 8, D & C Orange 4, FD & C Red 4, D & C Red 28, D & C Red 33, FD & C Red 40, and D & C Yellow 8, and said organic lakes such as FD & C Yellow 5 Lake, FD & C Yellow 6 Lake, Erythrosine Lake, D & C Yellow 10 Lake, Quinoline Yellow Lake, D & C Red 21 Lake, Carmoisine Lake, Ponceau 4R Lake, FD & C Blue 1 Lake, FD & C Blue 2 Lake, D & C Red 7 Ca. Lake, FD & C Red 40 Lake, D & C Red 27 Lake, Amaranth Lake, Patent Blue V Lake, and D & C Red 6 Ba Lake.

12. The tablet according to anyone of items 9 to 11, wherein said substance has a main absorption peak falling in the region UVB (280 to 315 nm), UVA (315 to 400 nm) or 400 nm to 450 nm, and/or

said substance is selected from the inorganic pigments titanium dioxide, iron oxide and/or zinc oxide, and from the water soluble dyes FD & C Yellow 5 (Tartrazine), FD & C Yellow 6 (Sunset Yellow), Quinoline Yellow, D & C Yellow 10 and/or FD & C Red 40, more preferably said substance is iron oxide, titanium dioxide, FD & C Red 40, FD & C Yellow 5 (Tartrazine) and/or FD & C Yellow 6 (Sunset Yellow).

13. The tablet as defined in anyone of items 9 to 12 for use in the treatment of a central nervous system disease, in particular schizophrenia.

14. A package including one or more pharmaceutical compositions comprising brexpiprazole dihydrate, wherein said package comprises a packaging material, which at least partially, preferably entirely encloses said one or more pharmaceutical compositions and which is capable of blocking, absorbing and/or reflecting UV exposure up to a wavelength of 450 nm.

15. The package according to item 14, wherein the packaging material comprises aluminum foil and/or polyvinyl chloride (PVC) or polyvinylidene chloride (PVDC) selected to block, absorb and/or reflect UV exposure up to a wavelength of 450 nm.

16. The package according to item 14, wherein the packaging material comprises borosilicate glass, preferably borosilicate glass comprising at least 0.2% Fe₂O₃, such as at least 0.5% Fe₂O₃. The glass is preferably of type 1 and acceptable as a pharmaceutical packaging.

17. The package according to any one of items 14 to 16 including an injectable pharmaceutical composition.

18. A process for producing a pharmaceutical composition, preferably an injectable pharmaceutical composition, comprising brexpiprazole dihydrate, wherein during a manufacturing step a composition comprising brexpiprazole dihydrate is protected against exposure to UV irradiation up to a wavelength of 450 nm to reduce or prevent UV-induced brexpiprazole impurities and in particular dimerization of brexpiprazole during its production, with this brexpiprazole dimer being characterized by anyone of the following characteristics (i) to (iv), respectively alone or in combination, preferably characteristic (iv) and, optionally, additionally any one of characteristics (i) to (iii):

(i) UV-chromatographic RRT of 0.92±0.02

(ii) LC-MS molecular weight [M+H]+=867±1,

(iii) chemical formula C₅₀H₅₄N₆O₄S₂,

(iv) chemical structure of Formula I:

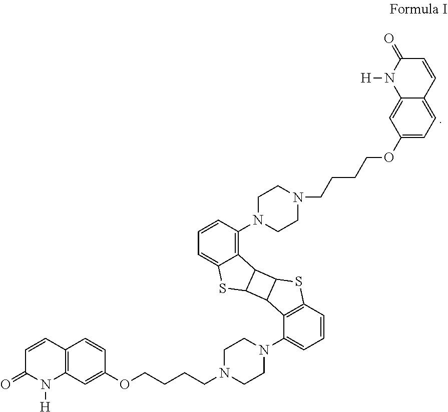 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

in particular wherein the manufacturing step is selected from dispensing of brexpiprazole dihydrate, preparing a bulk mixture comprising brexpiprazole duhydrate and filling an injectable pharmaceutical composition comprising brexpiprazole dihydrate into vials.

19. A manufacturing system adapted for carrying out a process according to item 18.

20. A method of evaluating the suitability of a batch of brexpiprazole dihydrate for the preparation of a pharmaceutical composition, preferably an injectable pharmaceutical composition, comprising the steps of:

(1) providing a sample of the batch of brexpiprazole dihydrate;

(2) determining the presence or absence of a brexpiprazole dimer that is characterized by anyone of the following characteristics (i) to (iv), respectively alone or in combination, preferably characteristic (iv) and, optionally, additionally anyone of characteristics (i) to (iii):

(i) UV-chromatographic RRT of 0.92±0.02

(ii) LC-MS molecular weight [M+H]+=867±1,

(iii) chemical formula C₅₀H₅₄N₆O₄S₂,

(iv) chemical structure of Formula I:

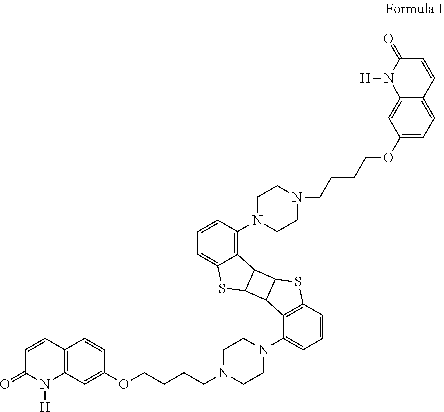 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

(3) determining that the batch is suitable for the preparation of a pharmaceutical composition if there is essentially no amount of the brexpiprazole dimer as defined in item 20 (2) is detectable.

In a preferred embodiment, the pharmaceutical composition is an injectable pharmaceutical composition/injectable preparation.

21. Use of a brexpiprazole dimer that is characterized by anyone of the following characteristics (i) to (iv), respectively alone or in combination, preferably characteristic (iv) and, optionally, additionally anyone of characteristics (i) to (iii):

(i) UV-chromatographic RRT of 0.92±0.02

(ii) LC-MS molecular weight [M+H]+=867±1,

(iii) chemical formula C₅₀H₅₄N₆O₄S₂,

(iv) chemical structure of Formula I:

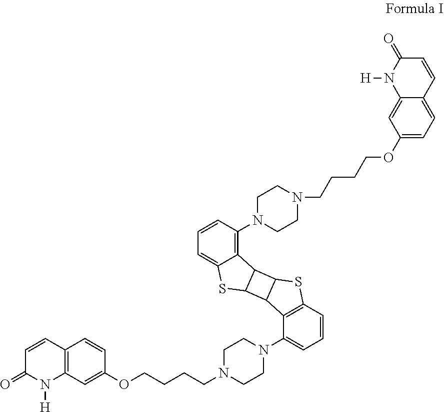 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

for evaluating the suitability of a batch of brexpiprazole dihydrate for the preparation of a pharmaceutical composition, preferably an injectable pharmaceutical composition, wherein said batch of brexpiprazole dihydrate is suitable for the preparation of a pharmaceutical composition if there is essentially no brexpiprazole dimer as defined above in this item present.

22. A pharmaceutical composition comprising brexpiprazole, which is defined by at most 2.0% w/w of brexpiprazole dimer relative to the total amount of brexpiprazole, wherein the brexpiprazole dimer is characterized by anyone of the following characteristics (i) to (iv), respectively alone or in combination, preferably characteristic (iv) and, optionally, additionally anyone of characteristics (i) to (iii):

(i) UV-chromatographic RRT of 0.92±0.02

(ii) LC-MS molecular weight [M+H]+=867±1,

(iii) chemical formula C₅₀H₅₄N₆O₄S₂,

(iv) Chemical structure of Formula I

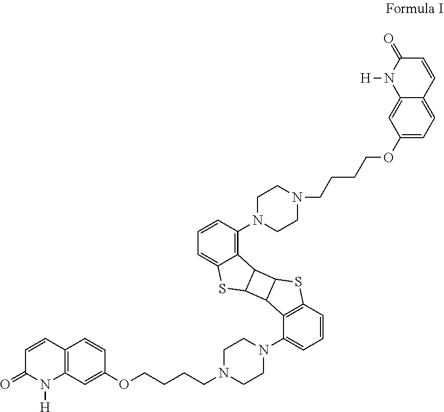 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

For measurement of item (i) and (ii) reference can be made to item 8 above.

In a preferred embodiment, the pharmaceutical composition is an injectable pharmaceutical composition.

23. A pharmaceutical composition comprising brexpiprazole dihadrate, wherein the amount of the brexpiprazole dimer of formula I in the pharmaceutical composition is at most 1.00% w/w relative to the amount of brexpiprazole in said same composition, more preferably it is at most 0.50%, such as at most 0.20%, for example at most 0.10%.

The amount of the compound of Formula I is determined by applying HPLC. If the amount is below 0.020% w/w, the amount of the compound of Formula I is determined by applying LC-MS.

24. The pharmaceutical composition according to item 22 or 23, capable of forming an aqueous suspension for parenteral administration, preferably for intramuscular or subcutaneous administration, most preferably for intramuscular injection.

In a preferred embodiment, the pharmaceutical composition is a sustained-release injectable pharmaceutical composition.

25. The pharmaceutical composition according to item 24, being an aqueous suspension.

26. The aqueous suspension according to item 25, wherein the aqueous suspension is in the form of an injectable preparation, preferably being ready to use.

27. The aqueous suspension according to item 25 or 26, comprising one or more pharmaceutically acceptable excipients in solid, liquid, or dissolved state.

28. The aqueous suspension according to any of items 25 to 27, comprising the brexpiprazole dihydrate in an amount of 0.5 wt.-% to 18.0 wt.-%, preferably 5 wt.-% to 15 wt.-%, based on the total weight of the aqueous suspension.

29. Injectable pharmaceutical preparation, comprising the aqueous suspension according to any one of items 25 to 28.

30. The injectable pharmaceutical preparation according to item 29, being ready to use.

31. Vial or prefilled syringe comprising the pharmaceutical composition according to any one of items 22 to 24, the aqueous suspension according to any one of items 25 to 28, or the injectable pharmaceutical composition according to item 29 or 30.

32. The pharmaceutical composition as defined in any one of items 22 to 24, the aqueous suspension as defined in any one of items 25 to 28, the injectable pharmaceutical composition as defined in item 29 or 30, or the vial or prefilled syringe as defined in item 31, which provides a unit dose, preferably providing a therapeutically effective blood concentration of brexpiprazole, for the treatment of a central nervous system disease, in particular schizophrenia.

33. A method of analyzing brexpiprazole dihydrate, the method comprising: (a) providing a sample of brexpiprazole dihydrate; and (b) quantifying an amount of compound of formula I

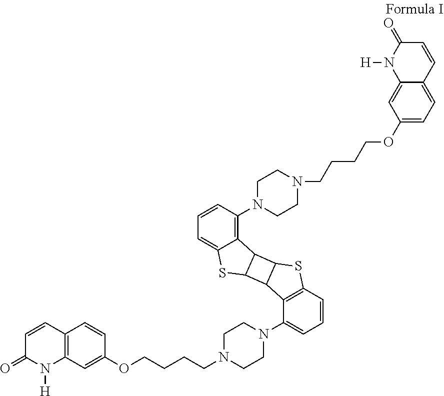 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

34. The method of item 33, wherein quantification is done by HPLC or NMR.

35. A method of analyzing a pharmaceutical composition comprising brexpiprazole dihydrate, the method comprising: (a) providing a sample of the pharmaceutical composition comprising brexpiprazole dihydrate; and (b) quantifying the amount of compound of formula I

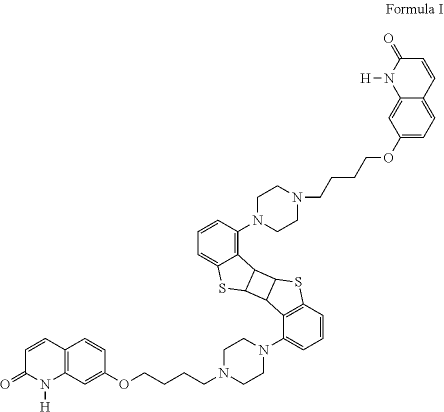 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

36. The method of item 35, wherein quantification is done by HPLC or NMR.

37. A method of classifying whether brexpiprazole dihydrate is suitable for release as a solid form of brexpiprazole to be used for the production of a pharmaceutical composition comprising brexpiprazole, the method comprising (a) providing a sample of the brexpiprazole dihydrate; (b) quantifying the amount of compound of formula I

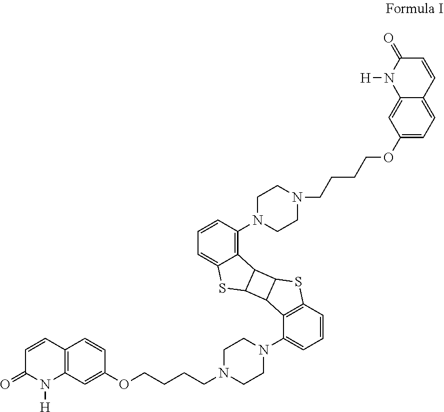 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

and (c) classifying brexpiprazole dihydrate as suitable for release if the amount of compound of formula I is at most 2.0% w/w relative to the total amount of brexpiprazole, preferably at most 1.0% w/w, such as at most 0.5% w/w, for example at most 0.2% w/w.

38. The method of item 37, wherein quantification is done by HPLC or NMR.

39. A method of classifying whether a pharmaceutical composition comprising brexpiprazole dihydrate is suitable for release, the method comprising (a) providing a sample of the pharmaceutical composition comprising brexpiprazole dihydrate; (b) quantifying the amount of compound of formula I

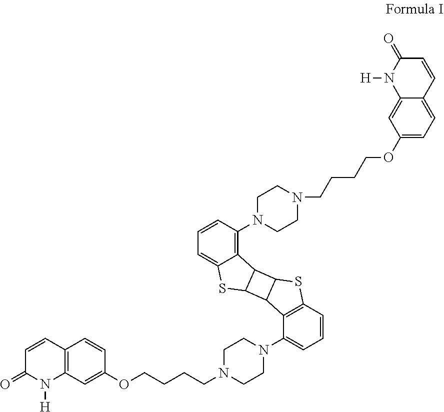 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

and (c) classifying the pharmaceutical composition comprising brexpiprazole dihydrate as suitable for release if the amount of compound of formula I is at most 2.0% w/w relative to the total amount of brexpiprazole, preferably at most 1.0% w/w, such as at most 0.5% w/w, for example at most 0.2% w/w.

40. The method of item 39, wherein quantification is done by HPLC or NMR.

41. Compound of formula I

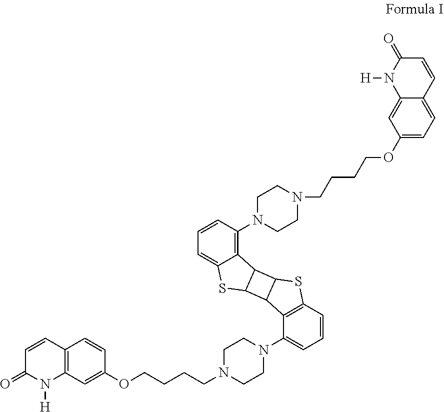 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

### Definitions

The term “brexpiprazole” means 7-[4-(4-benzo[b]thiophene-4-yl-piperazine-1-yl)butoxy]-1H-quinoline-2-one respectively the compound of Formula I itself, or a salt thereof. Furthermore, unless specified otherwise by the use of a distinct indication, the term “brexpiprazole” used herein means any physical form including amorphous or crystalline form, and any polymorphic form. The skilled person will appreciate that a reference to “brexpiprazole” further defined by powder characteristics, such as particle size parameters, means a reference to a composition consisting of solid brexpiprazole particles having the defined powder characteristics, such as the indicated particle size distribution.

The skilled person will appreciate that a reference to an excipient, such as a filler, further defined by powder characteristics, such as particle size parameters, means a reference to a composition consisting of solid excipient, such as filler, particles having the defined powder characteristics, such as the indicated particle size distribution.

The specifically indicated term “brexpiprazole anhydrate” as used herein refers to the crystalline form I of brexpiprazole anhydride, disclosed as “anhydride” in WO 2013/162046 A1, which is characterized by having a PXRD comprising reflections at 2-theta angles of 6.8°, 10.0°, 10.8°, 14.5°, 14.9°, 17.4°, 19.2°, 20.3°, 21.3° and 23.2° when measured by copper Kα_{1,2} radiation through a monochromator at a wavelength of 0.15418 nm. The crystalline form I of brexpiprazole anhydride can be prepared according to comparative example 1 of WO 2013/162046 A1.

Further, the specifically indicated term “brexpiprazole hydrate” as used herein refers to the crystalline form of brexpiprazole disclosed as “hydrate” in WO 2013/162046 A1 which is characterized by having a PXRD comprising reflections at 2-Theta angles of 7.7°, 9.4°, 11.8°, 18.9° and 24.0°, and preferably further peaks at 2-Theta angles of 5.7°, 8.1°, 8.8°, 10.7°, 12.6°, 13.6°, 13.9°, 15.0°, and 15.6°, when measured by copper Kapha_{1,2} radiation through a monochromator at a wavelength of 0.15418 nm.

The term “brexpiprazole dihydrate” as used herein refers to the crystalline form of brexpiprazole disclosed as “dihydrate” in WO 2013/162046 A1 which is characterized by having a PXRD comprising reflections at 2-Theta angles of 8.1°, 8.9°, 15.1°, 15.6° and 24.4°, and preferably further peaks at 2-Theta angles of 11.6°, 12.2°, 14.0°, 16.3°, 18.1°, 18.4°, 18.9° and 19.5°, when measured by copper Kalpha_{1,2} radiation through a monochromator at a wavelength of 0.15418 nm. Brexpiprazole dihydrate can have a water content according to Karl Fischer of from 6.5 to 8.8 wt. %.

The term “brexpiprazole dimer” as used herein denotes a brexpiprazole dimer that is characterized by anyone of the following characteristics (i) to (iv), respectively alone or in combination, preferably characteristic (iv) and, optionally, additionally any one of characteristics (i) to (iii):

(i) UV-chromatographic RRT of 0.92±0.02

(ii) LC-MS molecular weight [M+H]+=867±1,

(iii) chemical formula C₅₀H₅₄N₆O₄S₂,

(iv) chemical structure of Formula I:

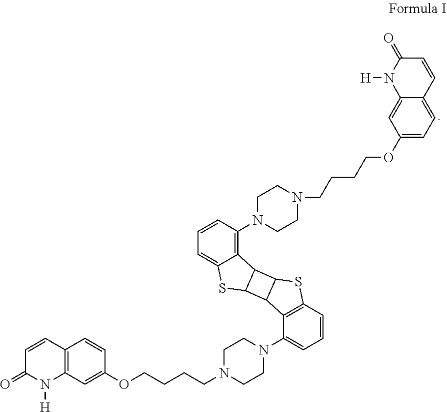 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

More preferred, the term “brexpiprazole dimer” as used herein denotes a brexpiprazole dimer that is characterized by a chemical structure of Formula I. (characteristic (iv)).

The expression “means capable of preventing exposure of brexpiprazole to UV light comprising wavelength of up to 450 nm”, or the capacity or feature “to block, absorb and/or reflect UV exposure in a wavelength region up to 450 nm” means that the brexpiprazole active ingredient is prevented from being substantially exposed to the corresponding critical UV wavelength region. In practice, the said substantially reduced critical UV exposure can be measured by standard transmission tests using a spectrophotometer, for instance as described corresponding pharmacopeiae (e.g. Ph. Eur.), such as in the light transmission test described in USP 37 <671> for containers or likewise adapted for measuring the respectively given substance or material. A suitable substantially reduced critical UV exposure, by virtue of blocking, absorption and/or reflection, is typically achieved if, in such reference spectroscopic light transmission tests the chosen substance or material or substance/material arrangement—when measuring the respective substance or material itself at the ultimately used concentration—exhibits light transmission of at most 70%, and in more preferred options at most 60%, at most 50%, at most 40%, at most 30%, at most 20%, at most 10% or at most 5% in the whole relevant UV wavelength region up to 450 nm.

As used herein, batch (or lot) refers to a specific quantity of material produced by a process or a series of processes to a final homogeneous state with specified limits and identified by a batch number and a material number. In the case of continuous production a batch may correspond to a defined fraction of the production. The batch size may be defined either by a fixed quantity or the amount produced in a fixed time interval. The batch can ultimately form or separated into a desired quantity of tablets according to the present invention, typically at least ten.

Subsequent batches can be identified via batch numbers and information on the batch production history. An example for five subsequent batches of “D=brexpiprazole dihydrate” is the uninterrupted sequence D, D, D, D, D, where no batch of brexpiprazole with a different end product “A=brexpiprazole anhydrate” or “D/A=Mixture of brexpiprazole dihydrate and brexpiprazole anhydrate” intervenes the sequence.

The term “direct compression” used herein means blending of active pharmaceutical ingredient (i.e. brexpiprazole API), with other ingredients (pharmaceutically acceptable excipients) and direct compaction of the resultant mixture. In direct compression, a dry formulation containing the API brexpiprazole and further ingredients are typically processed by applying a sufficient force by the punches of a tablet press on a powder to compact it into a tablet, notably a tablet core.

In “wet granulation”, granules are formed by the addition of a granulation liquid onto a powder bed including the active brexpiprazole API and further ingredients—usually requiring a polymer binder—which is under the influence of an impeller (shear and high-shear granulator), screws (twin screw granulator) or air (fluidized bed granulator). The agitation resulting in the system along with the wetting of the components within the formulation results in the aggregation of the primary powder particles to produce wet granules. The granulation liquid contains a volatile liquid—typically water, but also aqueous ethanol and isopropanol either alone or in combination—which is later removed by drying.

The term “dry granulation” used herein means a preparation or process wherein the dry formulation containing the active brexpiprazole API and further ingredients for a pharmaceutical dosage form are processed without using a liquid where granules are formed by compaction or densifying the powders, for instance compacting into compacts with roller compactor or tableting machine by using slugging tooling or regular tableting tooling and subsequently milling or crushing or otherwise sizing these compacts into dry granulate. The term “dry granulation” may also include moisture-activated dry granulation with strongly limited amount of liquid used (e.g. up to 10% and especially up to 5% water or aqueous alcohol such as ethanol or isopropanol liquid per formulation batch).

The term “coating” used herein means typically an outer coating. It can however also mean an intermediate coating, such as an intermediate layer of a tablet which is placed between a tablet core and an outer coating layer.

The terms “immediate release” (or its abbreviated term “IR”) and “immediate release tablet” corresponds to the definition provided in European Pharmacopeia 6.0, part 01/2008: 1502 as relating to “conventional-release dosage forms” or “immediate-release dosage forms” in the form of a tablet showing a release of the active substance (i.e. brexpiprazole API), which is not deliberately modified by a special formulation design and/or manufacturing method, thereby being distinct from “modify-release”, “prolong-release”, “delayed-release” and “pulsatile-release” dosage forms as defined in European Pharmacopeia 6.0, part 01/2008: 1502. More specifically, “immediate release” or “IR” can mean a release quantity of API of at least 75%, preferably at least 80% within a defined time, such as 60 min or typically 45 min or less, as determined according to Ph. Eur. 5.17.1.

In general, the term “sustained release” refers to a continuous release of an active pharmaceutical ingredient (API) over an extended period of time after administration of said API, thus providing a prolonged therapeutic effect throughout the release period. In particular, within the meaning of the present invention the term “sustained release” denotes that there is a continuous release of the crystalline brexpiprazole of the present invention after administration thereof to a subject in need thereof (i.e. a patient), to the effect that during a time period of at least one week after administration a therapeutically effective concentration of said brexpiprazole in the blood of the patient is maintained and thus can be determined. The respective blood concentration of brexpiprazole in order to be therapeutically effective may depend on the respective condition that is to be treated (e.g. treatment or prevention of relapse of schizophrenia, bipolar disorder, or depression) and is known to a person skilled in the art.

The term “essentially” used herein means at least 90%, preferably at least 95% and more preferably at least 98% of the indicated reference (in wt. % if a material is referred to).

The term “essentially no brexpiprazole dimer is present/is detectable” or “essentially no amount of brexpiprazole dimer is present” or “reduced or even prevented generation of UV-induced brexpiprazole dimer” or “reduced or prevented amount of brexpiprazole dimer” as used herein means that the amount of a brexpiprazole dimer as defined elsewhere herein, preferably of a brexpiprazole dimer exhibiting the chemical structure of Formula I

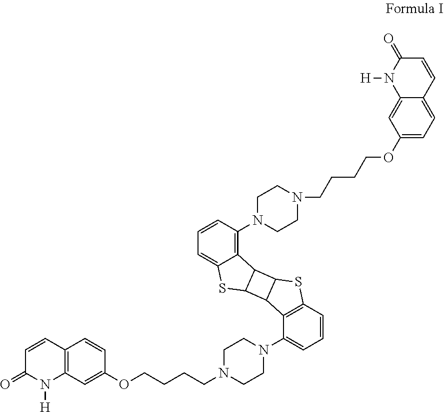 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

in a composition is at most 1.00% w/w relative to the amount of brexpiprazole in said same composition, more preferably it is at most 0.50%, such as at most 0.20%, for example at most 0.10%. The amount of the brexpiprazole dimer, preferably of the brexpiprazole dimer of Formula I, is determined by applying HPLC. If the amount is below 0.020% w/w, the amount is determined by applying LC-MS.

The lower limit of detection for the amount of the brexpiprazole dimer, preferably of the brexpiprazole dimer of Formula I, can e.g., be 0.005% w/w relative to the amount of brexpiprazole in said same composition.

In the context of the present invention the term “RRT” means relative retention time, i.e. the retention time of a compound in a C-18 reverse phase HPLC column relative to the peak representing the compound brexpiprazole. Numbers smaller than 1.0 indicate a compound which elutes earlier than brexpiprazole, numbers larger than 1.0 indicate a compound which elutes later than brexpiprazole.

For the purpose of this invention, particle size distribution is determined as the percent volume at each particle size and measured by a laser diffraction method in the context of a circulating aqueous suspension. A Malvern Mastersizer 3000 laser diffraction analyzer equipped with a Hydro EV measurement cell is to be used.

For brexpiprazole about 50 mg of sample were filled into a 15 ml glass test tube. The sample was wetted with two drops of Tween 80 and vortexed. Then the thoroughly wetted sample was dispersed in about 7 mL of UHQ-water by vortexing until the particles were dispersed. The suspension was treated for 7 minutes in an ultrasonification bath at RT (Elma S50H). The well dispersed suspension was then measured in water by adding a sample from the center of the test tube containing the well dispersed suspension to a water-filled dispersion unit prefilled with 70 mL of water. The sample in the 70 mL dispersion unit was kept under stirring at 2000 rpm. More sample from the test tube was added, until a targeted obscuration range of 10-25 percent was achieved. The Malvern Mastersizer was operated using the following parameters:

Background time—8 s.

Measurement time—12 s.

Refractive index dispersant—1.33

Stirring unit—2000 U/min (as mentioned above).

External sonification—5 min (as mentioned above)

Internal sonification—no.

Waiting period—3 Minutes.

Diffraction method—Fraunhofer theory.

Analysis model—general purpose.

Sensitivity—normal

Blue laser is on, results are by volume distribution.

Measurement occurred after an optical alignment of the laser was done and after a background measurement was run. A measurement sequence consisted of eight individual measurements for which the mean value was represented as a histogram.

D90 as used herein means that 90% of the particles (based on volume) are smaller than or equal to the indicated size.

D50 as used herein means that 50% of the particles (based on volume) are smaller than or equal to the indicated size.

D10 as used herein means that 10% of the particles (based on volume) are smaller than or equal to the indicated size.

The term “injectable pharmaceutical composition” or “injectable pharmaceutical preparation” as used herein means a pharmaceutical composition or preparation that, optionally after additional preparation steps such as lyophilisation, reconstitution, and/or combining it with water such as water for injection, can be administered by injecting it into a subject. Usually, injection takes place by means of a syringe. Preferably, the injectable composition or injectable preparation is sterile.

In the present invention, the terms “injectable preparation”, “injectable composition” and “injectable formulation” are used interchangeable.

The injectable composition can be in form of an aqueous suspension.

If it is not necessary to carry out further additional preparation steps, the injectable composition or injectable preparation being in form of an aqueous suspension is ready to use. The expression “ready to use” means that said suspension can be used for administration immediately, with no need to subject said suspension to further preparation steps or the like, such as a lyophilisation and/or reconstitution step.

The term “therapeutically effective blood concentration” as used herein refers to the concentration of active agent (API; in the present invention, the active agent is brexpiprazole) in the blood, that produces a therapeutic effect.

### DETAILED DESCRIPTION OF THE INVENTION

The invention is described below in further detail by embodiments, without being limited thereto.

The present inventors have devised particular technical solutions for UV-specific protection for brexpiprazole dihydrate. Applying, respectively alone or in combination, (i) a substance selected as disclosed herein, in an amount sufficient to reduce or prevent UV-induced dimerization of brexpiprazole; and/or (ii) a packaging material enclosing the brexpiprazole dihydrate comprising pharmaceutical composition, preferably the brexpiprazole dihydrate comprising injectable pharmaceutical composition, has been found particularly effective in the prevention of UV-induced dimerization of brexpiprazole.

Besides dimerization of brexpiprazole other UV-induced brexpiprazole decomposition impurities can also be effectively controlled by the concept of the present invention. Brexpiprazole dihydrate has found to be specifically prone to a dimerization reaction under UV exposure, specifically encountering a problem of dimerization by exposure to a wavelength region of 450 nm or below, especially if exposed to UV irradiation including UVC from 200 to 280 nm, UVB from 280 to 315 nm and UVA from 315 to 400 nm.

The brexpiprazole dimer identified within the framework of the present invention can be characterized by anyone of the following characteristics (i) to (iv), respectively alone or in combination, preferably by characteristic (iv) and, optionally, additionally any one of characteristics (i) to (iii):

(i) UV-chromatographic RRT of 0.92±0.02

(ii) LC-MS molecular weight [M+H]+=867±1,

(iii) chemical formula C₅₀H₅₄N₆O₄S₂,

(iv) Chemical structure of Formula I

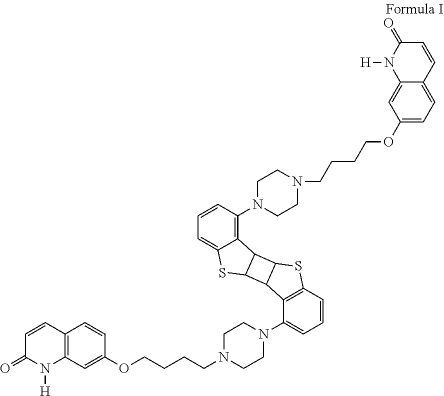 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

In particular, it has been found that if brexpiprazole is present in form of a dihydrate, the resulting UV-induced dimer is characterized (besides the characteristics listed above in (i) to (iii)) by a chemical structure of Formula I. Examples of pharmaceutical compositions that comprise brexpiprazole usually in form of a dihydrate are for instance injectable compositions.

Based on this surprising finding of the present invention, it is not only possible to provide pharmaceutical compositions that exhibit improved properties with regard to UV-induced dimerization, but also to provide a method for evaluating the suitability of brexpiprazole dihydrate for the preparation of a pharmaceutical composition.

Surprisingly, in a preferred embodiment a superior arrangement and structure of the pharmaceutical composition of the present invention has been found to significantly improve brexpiprazole stability against UV-induced dimerization, namely by the provision of a tablet comprising at least a tablet core, wherein the tablet core comprises brexpiprazole dihydrate in combination with a substance which is selected from the group consisting of inorganic pigments and organic pigments, solvent soluble dyes, water soluble dyes and organic lakes and which blocks, absorbs and/or reflects UV irradiation, particularly in the wavelength region(s) defined above. In such a direct association within the same tablet compartment of a tablet core, it appears that the selected substance can reduce or prevent UV-induced dimerization of brexpiprazole even more efficiently than when present in another compartment remote from the direct brexpiprazole microenvironment.

With regard to further pharmaceutical compositions, such as injectable pharmaceutical compositions, the principle of preventing UV-induced dimerization by addition of selected substance can be applied accordingly: In this case, the injectable pharmaceutical composition comprises brexpiprazole dihydrate in combination with a substance as disclosed above, with the proviso that the substance added for UV-protection is suitable as an excipient for an injectable pharmaceutical preparation.

Effective substances capable of reducing or preventing dimerization of brexpiprazole, in particular the dimerization of brexpiprazole to a dimer being characterized by anyone of the characteristics (i) to (iv), respectively alone or in combination, preferably characteristic (iv) and, optionally, additionally anyone of characteristics (i) to (iii), include, without being limited to, dyes and pigments showing UV absorption within the wavelength region of 450 nm or below and being selected from inorganic pigments such as red iron oxide, yellow iron oxide, black iron oxide, zinc oxide and titanium dioxide; organic pigments such as D & C Red 30, D & C Red 34 and D & C Red 36; solvent soluble dyes such as Green 6, D & C Red 17, D & C Violet 2, D & C Yellow 7, D & C Yellow 11, D & C Red 21, D & C Red 27, and D & C Orange 5; water soluble dyes such as D & C Green 5, FD & C Green 3 (Fast Green), FD & C Yellow 5 (Tartrazine), FD & C Yellow 6 (Sunset Yellow), Quinoline Yellow, D & C Yellow 10, FD & C Red 3 (Erythrosine), D & C Red 22, FD & C Blue 1 (Brilliant Blue), FD & C Blue 2 (Indigotine), FD & C Blue 3, Ponceau 4R, Carmoisine, Naphthol Blue lack, Amaranth, Patent Blue V, Black PN, Orange G, D & C Green 8, D & C Orange 4, FD & C Red 4, D & C Red 28, D & C Red 33, FD & C Red 40, and D & C Yellow 8; and organic lakes such as FD & C Yellow 5 Lake, FD & C Yellow 6 Lake, Erythrosine Lake, D & C Yellow 10 Lake, Quinoline Yellow Lake, D & C Red 21 Lake, Carmoisine Lake, Ponceau 4R Lake, FD & C Blue 1 Lake, FD & C Blue 2 Lake, D & C Red 7 Ca. Lake, FD & C Red 40 Lake, D & C Red 27 Lake, Amaranth Lake, Patent Blue V Lake, and D & C Red 6 Ba Lake. Preferred are the inorganic pigments red iron oxide, yellow iron oxide, black iron oxide and titanium dioxide, and the water soluble dyes FD & C Yellow 5, FD & C Yellow 6, Quinoline Yellow, D & C Yellow 10 and FD & C Red 40. Preferred are pigments and dyes which have their main absorption peak falling in the critical wavelength region up to 450 nm, in particular in the region UVB (280 to 315 nm), UVA (315 to 400 nm) or 400 nm to 450 nm. Also preferred are pigments and dyes which, besides protecting brexpiprazole dimerization, themselves have good light stability, such as iron oxide, titanium dioxide, FD & C Red 40, FD & C Yellow 5 (Tartrazine) and FD & C Yellow 6 (Sunset Yellow).

In another useful and effective embodiment, a tablet according to the present invention adopts a core/coating or core/shell structure where brexpiprazole dihydrate is in the tablet core while additionally paying attention to the ability of the inorganic pigments titanium dioxide and/or zinc oxide, in particular of titanium dioxide, present in a coating to effectively control blockage, absorption and/or reflection of UV irradiation and in particular in the above defined wavelength region(s), when present in sufficient coating thickness or dry coating mass as such, or when present in sufficient amount in relation to the core or the brexpiprazole in the core. Although not wishing to be bound by a certain theory, it may be believed that the inorganic pigments titanium dioxide and/or zinc oxide and particularly titanium dioxide, due to their inherent UV-specific absorption behaviour and by virtue of the defined core/shell arrangement, show highly effective blockage, absorption and/or reflection of UV irradiation in the above mentioned critical wavelength region where brexpiprazole has been found particularly sensitive to UV-induced impurity formation and especially dimerization.

It has been found that such additional control can be effectively accomplished in various ways. Preferred embodiments include:

According to a first option, a thickness or dry coating mass of the coating layer comprising titanium dioxide and/or zinc oxide, preferably titanium dioxide, is provided sufficient to minimize or prevent UV-induced brexpiprazole impurity generation and especially dimerization that could occur in the core. As an indication of a thickness sufficient for such protection, dry coating mass has been found to represent a good protection parameter, characterized by at least 6.7 wt. %, more effectively by at least 10 wt. % and further preferred by at least 11% or even at least 15 wt. % dry coating mass relative to the weight of the tablet core.

As a further option, considered independently or preferably in combination with the layer thickness or dry coating mass, titanium dioxide and/or zinc oxide (preferably titanium dioxide) itself is contained in the coating in an amount of at least 1 wt. %, preferably at least 2 wt. % and more preferably at least 3 wt. % relative to the weight of the tablet core. Another effective option was found to be a weight ratio of titanium dioxide and/or zinc oxide in the coating relative to the weight of brexpiprazole (preferably brexpiprazole dihydrate) present in the tablet core (TiO₂ and/or ZnO:brexpiprazole), when this ratio value is at least 0.2:1 or a higher TiO₂ and/or ZnO:brexpiprazole ratio. More effective and thus preferred is when the TiO₂ and/or ZnO:brexpiprazole ratio is in a range of 0.2-3:1, more preferably 0.2-2:1, even more preferably 0.3-1:1. A selection of titanium oxide in the present option is preferred.

By applying the technical concept according to the present invention, an amount of brexpiprazole dimer being characterized as defined elsewhere herein, preferably by a chemical structure of Formula I, in the tablet or in a further pharmaceutical composition can be controlled to a level of at most 1%, preferably at most 0.5% of the entire tablet weight when in a test the tablet is exposed to UV light with a total intensity of 1350 W·h/m². Furthermore, a total amount of UV-induced brexpiprazole impurities in the tablet can be controlled to a level of at most 2%, preferably at most 1% when in a test the tablet is exposed to UV light with a total intensity of 1350 W·h/m².

For the provision of one or more coating layers around the tablet core—optionally only a single outer coating layer is provided around the tablet core—any method for film coating, known in the field of the pharmaceutical technology, may be used; typically the coating is sprayed on the tablet cores as a suspension, the suspension being prepared either by mixing of single excipients or by using ready-made mixtures (e.g. Opadry). Besides or in addition to a consideration of the above described embodiments of a protection against UV-induced brexpipratole decomposition and dimerization, the one or more coating(s) may comprise at least one further additive suitable for preparing the coating layer(s). The at least one additive is preferably selected from the group of film-forming polymers, plasticizers, glidants, and anti-tacking agents, and pigments serving a desired function. Suitable film-forming polymer additives of the coating(s) may include, without being limited to, a polymer selected from polyvinyl alcohol, hydroxypropyl methylcellulose, hydroxypropyl cellulose, hydroxyethylcellulose and polymethacrylates. Suitable plasticizers may be selected from the group consisting of triethyl citrate, polyethylene glycol, propylene glycol, dibutyl sebacate, diethyl phthalate, dibutyl phthalate, glycerol monostearate, triacetin, and the like. Further suitable additives the one or more coating(s) include general purpose colorants and/or pigments, and/or antitacking agents, in particular talc. Further, dispersing agents may assist to disperse any colorants, pigments and/or minerals to be included into the coating(s).

It is preferred that all or essentially all of the brexpiprazole in the tablet is crystalline, preferably it is brexpiprazole dihydrate. The brexpiprazole dihydrate is for example obtainable as disclosed in WO 2013/162046 A1. Brexpiprazole dihydrate is preferably crystalline.

As disclosed elsewhere herein, the UV-induced dimer of brexpiprazole dihydrate is a dimer that is characterized by any one of the following characteristics (i) to (iv), respectively alone or in combination, preferably characteristic (iv) and, optionally, additionally anyone of characteristics (i) to (iii):

(i) UV-chromatographic RRT of 0.92±0.02,

(ii) LC-MS molecular weight [M+H]+=867±1,

(iii) chemical formula C₅₀H₅₄N₆O₄S₂,

(iv) Chemical structure of Formula I

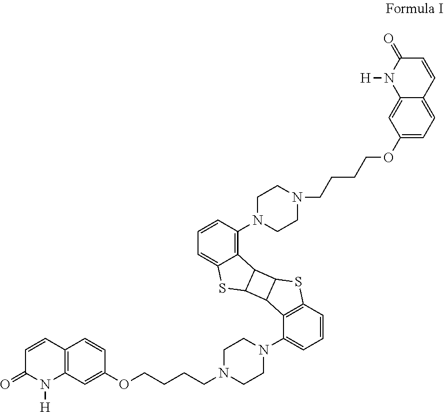 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

As was found in the present invention, brexpiprazole dihydrate is surprisingly significantly less stable than for instance brexpiprazole anhydrate towards exposure to UV-radiation. This means that it is more prone to UV-induced dimerization when compared to brexpiprazole anhydrate. Thus, stabilizing brexpiprazole dihydrate in a pharmaceutical composition is significantly more important than for other solid forms of brexpiprazole, such as brexpiprazole anhydrate. Consequently the preparation of pharmaceutical compositions comprising brexpiprazole dihydrate benefits from proper handling of brexpiprazole dihydrate during production of brexpiprazole dihydrate as a solid form of the active pharmaceutical ingredient brexpiprazole, it benefits from proper UV-protection during handling and/or storage and/or transport of brexpiprazole dihydrate, it benefits from proper UV-protection during the preparation of a pharmaceutical dosage form comprising brexpiprazole dihydrate and it benefits from proper UV-protection during storage and/or transport of a pharmaceutical dosage form comprising brexpiprazole dihydrate. Preferably proper UV-protection of brexpiprazole dihydrate is applied during all the above-mentioned steps.

The pharmaceutical composition comprising brexpiprazole dihydrate according to the present invention can be formulated as an oral dosage form, in a solid or a liquid form, as a depot formulation, or any other desired dosage form. Preferred are injectable pharmaceutical compositions. Particularly preferred, the injectable pharmaceutical compositions are designed as a sustained release dosage form.

A pharmaceutical composition according to the present invention can be prepared by known methods. Specifically in cases where a granule-containing capsule or a tablet having at least a core shall be provided, the preparation process may include direct compression, dry granulation or wet granulation.

As inactive ingredients which can be included within a pharmaceutical composition according to the present invention, at least one pharmaceutically acceptable excipient can be suitably selected, e.g., from the group consisting of fillers, glidants, disintegrants, surfactants and lubricants, and optionally in case of using wet granulation and optionally also for dry granulation, polymer binders.

Generally and independent from applying other UV exposure protection concepts disclosed herein, and as a further possibility (optionally in combination with the above described possibilities and embodiments) to improve stability against UV-induced dimerization and impurity generation in view of potential exposure in the critical wavelength region, the packaging material preferably is selected to block, absorb and/or reflect UV exposure up to a wavelength of 450 nm. Accordingly, this is suitably accomplished by selecting appropriate packing material having the capacity of blocking, absorbing and/or reflecting UV exposure up to a relevant wavelength region of 450 nm, particularly in the UVB and/or UVA region(s). Preferably, the packaging material comprises aluminum foil and/or polyvinyl chloride (PVC) or polyvinylidene chloride (PVDC) correspondingly selected to block, absorb and/or reflect UV exposure up to a wavelength of 450 nm, or is made of a combined aluminum/polymer foil. It is also possible that the packaging material comprises or essentially consists of glass, such as brown glass, exhibiting the above characteristics.

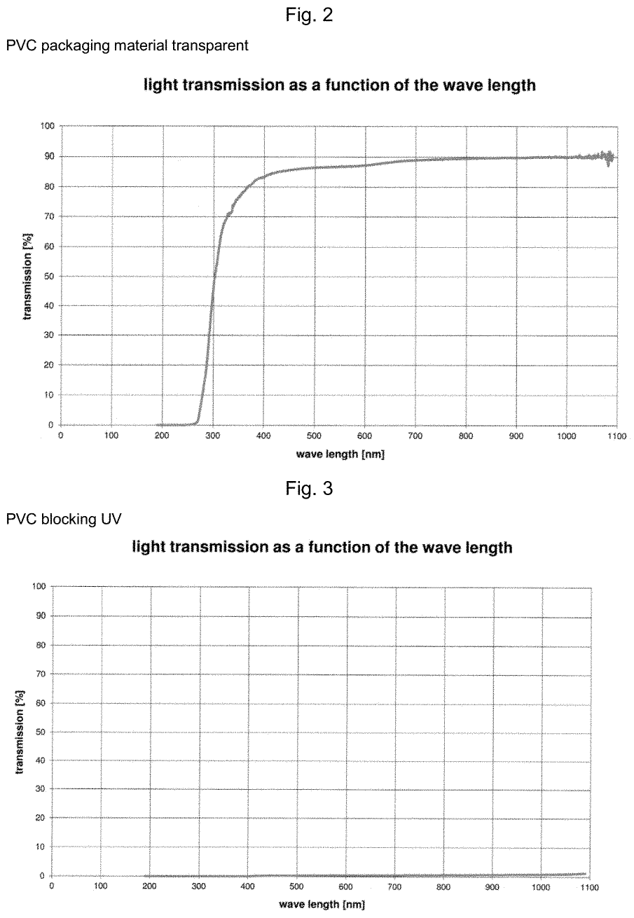

According to another aspect of the present invention a process and a manufacturing system for producing a pharmaceutical composition comprising brexpiprazole dihydrate is provided, wherein—thanks to the findings of the present invention—during at least relevant, preferably during all manufacturing steps as well as intermittent steps any composition, including intermediate products, which respectively comprise brexpiprazole dihydrate is protected against exposure to UV irradiation up to a wavelength of 450 nm and especially in the UVB and/or UVA region(s) to reduce or prevent UV-induced brexpiprazole impurities and in particular dimerization of brexpiprazole during its production. This can be accomplished by suitable shielding or protection means which enclose said brexpiprazole compositions or intermediate products and which are capable of blocking, absorbing and/or reflecting UV exposure up to a wavelength of 450 nm, especially in the wavelength regions disclosed herein. For illustration purposes, reference is made to FIGS. 2 and 3 which show light transmissions depending on the wavelength of two distinct polymer packaging films or foils. While both exemplified polymer packaging films are basically made of PVC (respectively obtained from Klöckner Pentaplast, Germany), they differ in their UV specific wavelength absorption and thus UV blocking effect: while normal PVC is transparent in the critical UV wavelength region, in particular UVB (280 to 315 nm) and UVA (315 to 400 nm), as shown in FIG. 2, the specifically selected PVC one for use in the present preferred embodiment does show substantial blocking in the UV-specific wavelength region including UVB and UVA (FIG. 3). Accordingly, even if the pharmaceutical composition themselve may not be protected from UV-induced impurity formation, the specific UV-induced brexpiprazole dimer can be significantly reduced when packed in the selected packaging materials. And protection from UV-induced dimer formation can be further enhanced if both protection concepts are combined, e.g. if the pharmaceutical composition is a tablet, or an injectable pharmaceutical composition, the protection by the tablets or injectable pharmaceutical composition themselves as described above combined with protection by a selected packaging material.

The pharmaceutical composition according to the present invention is particularly useful in the treatment of a central nervous system disease, particularly for the treatment of schizophrenia, or other CNS disorders.

As already disclosed elsewhere herein, brexpiprazole can preferably be present as brexpiprazole dihydrate. While the stability of an API, such a brexpiprazole, is an issue of general importance, it was surprising based on the experience with other solid form of brexpiprazole that brexpiprazole dihydrate posed particular challenges. This is because brexpiprazole dihydrate is significantly more prone to UV-induced dimerization than e.g. brexpiprazole anhydrate. Even more surprisingly, brexpiprazole dihydrate formed a dimer which was structurally different from the UV-induced dimer generated from brexpiprazole anhydrate. This is thus a very rare case where chemical reactivity of a compound, in this case brexpiprazole, is affected both quantitatively as well as qualitatively by the particular crystal form that the compound is in.

As pharmaceutical compositions that comprise brexpiprazole dihydrate have been described to have particular therapeutic utility, in particular for injectable compositions, there is a need for such pharmaceutical compositions to exhibit improved performance and use attributes in particular with respect to improved stability during preparation and storage. This improvement is conferred by reducing or preventing the UV-induced dimerization of brexpiprazole dihydrate.

Conferring improved stability to brexpiprazole dihydrate can be achieved by reducing or preventing UV-induced dimerization thereof. This, in turn, can be achieved on the one hand by protecting brexpiprazole dihydrate by combining it with a substance that is present in an amount sufficient to reduce or prevent UV-induced dimerization of brexpiprazole, preferably UV-induced dimerization of brexpiprazole dihydrate, wherein the resulting dimer is being characterized as defined elsewhere herein, preferably by a chemical structure of Formula I as disclosed elsewhere herein. On the other hand, this can be achieved by protecting the pharmaceutical composition comprising the brexpiprazole dihydrate with a package that comprises a packaging material and that contains a pharmaceutical composition, preferably an injectable composition, comprising brexpiprazole, wherein said packaging material is capable of blocking, absorbing, and/or reflecting UV exposure up to a wavelength of 450 nm. The respective substances and packaging materials are disclosed elsewhere herein. In a preferred embodiment, the package comprises type I glass, or is a glass container, in particular if the pharmaceutical composition is an injectable pharmaceutical composition. More preferably, the glass is brown glass. Also containers composed of plastic having the desired UV-protecting characteristics are suitable. Special materials are available for parenteral products and may be used, e.g. triple-layer materials of a layer of polyamide sandwiched between two COP (cyclic olefin polymer) layers.

Thus, the present invention also refers to a package including one or more pharmaceutical compositions comprising brexpiprazole dihydrate, wherein said package comprises a packaging material, which at least partially, preferably entirely encloses said one or more pharmaceutical compositions and which is capable of blocking, absorbing and/or reflecting UV exposure up to a wavelength of 450 nm. If the pharmaceutical composition is an injectable composition, it is preferred that the package is type I glass, such as brown borosilicate glass.

The resulting, thus-protected pharmaceutical composition comprising brexpiprazole dihydrate is defined by a reduced or prevented amount of brexpiprazole dimer that is characterized as disclosed elsewhere herein, preferably by a chemical structure of Formula I:

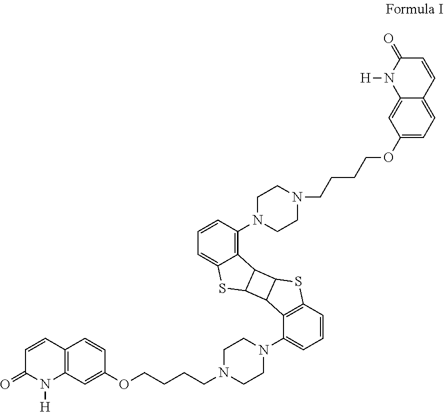 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

By taking measures to protect pharmaceutical compositions comprising brexpiprazole dihydrate from UV-induced dimerization, the amount of brexpiprazole dimer exhibiting the characteristics as disclosed elsewhere herein in the pharmaceutical composition is at most 1.00% w/w relative to the amount of brexpiprazole dihydrate in said same composition, more preferably it is at most 0.50%, such as at most 0.20%, for example at most 0.10%.

The pharmaceutical composition can be present in various forms, such as in an uncompressed form. Suitable uncompressed forms are known to a person skilled in the art. Examples of such suitable uncompressed forms are a liquid, e.g. a suspension, or a powder. The choice of pharmaceutically acceptable excipient(s) that is (are) present in the pharmaceutical composition is also dependent on the respective form (or state) of said composition. For instance, if the pharmaceutical composition is intended to be in form of a powder, a person skilled in the art will choose the pharmaceutically acceptable excipients that are suitable for this purpose.

Alternatively, or additionally, the pharmaceutical composition can be present in solid state. This solid state can be an uncompressed or a compressed solid state. Dependent on whether compression is carried out, excipients may have to be present that render the pharmaceutical composition compressible.

Examples of pharmaceutical compositions in solid state are tablets, capsules, powders, or lozenges. Of these, tablets are an example of a compressed, solid form.

Particular examples of pharmaceutical compositions in uncompressed solid forms are powders, capsules, and lyophilisates.

A “lyophilisate” is the result of a process called lyophilisation. Lyophilization, also referred to as “freeze drying”, is a process in which water or another solvent is frozen, followed by the removal of said water/solvent from the sample, by sublimation and then by desorption. During lyophilization, the moisture content of the sample is reduced to such a low level that for instance does not support chemical reactions. Lyophilization thus is particularly useful in formulating APIs that are thermolabile and/or unstable in water.

It is known to a person skilled in the art that different formulation techniques may require specific pharmaceutically acceptable excipients, dependent on the respective demands of the applied technique(s) and/or the envisaged final product or final dosage form, respectively. For instance, if a lyophilisate shall be prepared, it is one important aspect to provide a lyophilisate that can be reconstituted without any problems. Therefore, excipients have to be added that provide for an adequate structure to the lyophilisate, with this adequate structure allowing a proper reconstitution. Accordingly, if the pharmaceutical composition of the present invention is in form of a lyophilisate, one or more bulking agents, such as sugars (e.g. mannitol, lactose, sucrose, trehalose, sorbitol, glucose, raffinose, or a combination thereof), amino acids (e.g. arginine, glycine, or histidine, or a combination thereof), or polymers (e.g. dextran or polyethylene glycol), are preferably present. In a more preferred embodiment, said bulking agent(s) is (are) mannitol, sorbitol, sucrose, or a combination thereof. Bulking agents, and in particular the above bulking agents, provide for a proper structure of the lyophilizate (cake).

The pharmaceutical composition of the present invention, being in form of a lyophilisate, is suitable for reconstitution to form an aqueous suspension for parenteral administration, preferably for intramuscular or subcutaneous administration, most preferably for intramuscular injection.

Reconstitution can for instance be carried out by adding a suitable aqueous solution, preferably water for injection, to the lyophilisate.

In a further aspect, the pharmaceutical composition of the present invention that is not in form of a lyophilisate, is capable of forming an aqueous suspension for parenteral administration, preferably for intramuscular or subcutaneous administration, most preferred for intramuscular injection.

Upon reconstitution of said pharmaceutical composition that is suitable for reconstitution, or of said pharmaceutical composition that is capable of forming an aqueous suspension for parenteral administration, with an aqueous solution or water for injection, an aqueous suspension is obtained. In an optional embodiment, additionally further pharmaceutically acceptable excipients can be added.

The aqueous suspension is obtainable or can be obtained by combining brexpiprazole dihydrate, or the pharmaceutical composition according to the present invention comprising brexpiprazole dihydrate, with an aqueous solution or water. Optionally, further pharmaceutically acceptable excipient(s) is (are) added. These pharmaceutically acceptable excipients can be present in solid, liquid, or dissolved state.

In one embodiment, the brexpiprazole, preferably brexpiprazole dihydrate, is present in the aqueous suspension in an amount of 0.5 wt.-% to 18.0 wt.-%, preferably 5 wt.-% to 15 wt.-%, based on the total weight of the aqueous suspension.

In a preferred aspect, said brexpiprazole dihydrate or pharmaceutical composition comprising brexpiprazole dihydrate as defined in the present invention, being present in an aqueous suspension, is combined with water, preferably with water for injection, and optionally further pharmaceutically acceptable excipients.

In a further preferred embodiment, said aqueous suspensions are in the form of a sterile injectable preparation, with this sterile injectable preparation preferably being ready to use. It is particularly preferred that said aqueous suspensions are ready to use for parenteral administration, e.g. without the aqueous suspensions being firstly lyophilized and subsequently reconstituted.

In a further aspect, the present invention relates to a vial or prefilled syringe containing the pharmaceutical composition comprising brexpiprazole dihydrate according to the present invention, the aqueous suspension according to the present invention, or the injectable preparation according to the present invention.

In a preferred embodiment, the vial or prefilled syringe provides a unit dose, preferably provides an effective blood concentration of brexpiprazole for the treatment of a central nervous system disease, in particular schizophrenia, for at least one week.

In an additional preferred embodiment, the unit dose provides a unit dose of 10 mg to 90 mg brexpiprazole.

As disclosed elsewhere herein, it is also possible that the pharmaceutical composition is an injectable preparation, such as an aqueous suspension. Usually, in injectable preparations, brexpiprazole is present in its dihydrate form. Preferably, said injectable preparations or injectable compositions are sterile.

A sterile injectable formulation of the present invention can be produced, for example, as described in test example 4, examples C, D, E and F of CA 2871398 A1. A sterile injectable formulation of the present invention can also be produced, for example, as described in test example 5, examples G-1, G-2, G-3, G-4, G-5 and G-6 of CA 2871398 A1. A sterile injectable formulation of the present invention can also be produced, for example, as described in test example 5, examples H-1, H-2, H-3, H-4, H-5 and H-6 of CA 2871398 A1. A sterile injectable formulation of the present invention can also be produced, for example, as described in test example 5, examples I-1, I-2, I-3, I-4, I-5 and I-6 of CA 2871398 A1. A sterile injectable formulation of the present invention can also be produced, for example, as described in test example 5, examples J-1, J-2, J-3, J-4, J-5 and J-6 of CA 2871398 A1.

Example 2 of CA 2871398 A1 discloses a particularly preferred manner for the production of a sterile injectable formulation of the present invention.

In a further aspect, the present invention relates to a method of evaluating the suitability of a batch of brexpiprazole dihydrate for the preparation of a pharmaceutical composition, preferably an injectable composition, comprising the steps of:

(1) providing a sample of the batch of brexpiprazole dihydrate;

(2) determining the presence or absence of a brexpiprazole dimer that is characterized as disclosed elsewhere herein, preferably by the chemical structure of Formula I:

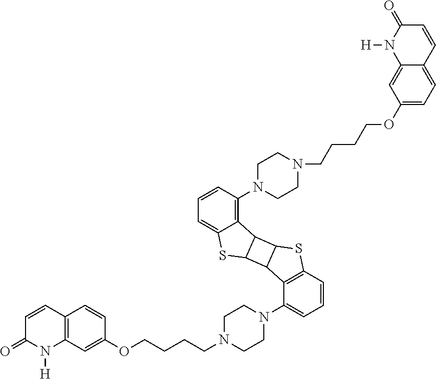 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

(3) determining that the batch is suitable for the preparation of a pharmaceutical composition if the amount of brexpiprazole dimer exhibiting the characteristics as disclosed elsewhere herein, preferably the amount of compound of formula I, in the tested batch is at most 1.00% w/w relative to the amount of brexpiprazole dihydrate in said tested batch, more preferably it is at most 0.50%, such as at most 0.20%, for example at most 0.10%. Preferably there is essentially no amount of the brexpiprazole dimer that is characterized as disclosed elsewhere herein, preferably that is characterized by the chemical structure of Formula I, detectable.

Typically, the size of the sample of step (1) is such that the presence or absence of the brexpiprazole dimer of Formula I can be determined. Preferably, more than one sample, for example two or three or more samples of a batch, are tested by determining the presence or absence of the specific brexpiprazole dimer.

In order to determine the presence or absence of a brexpiprazole dimer that exhibits characteristics as defined above, preferably a structure of Formula I, in step (2), any suitable method that is known to a person skilled in the art can be applied. Preferably, the presence or absence of a compound exhibiting the characteristics defined above is determined by applying HP-LC or LC-MS, as disclosed elsewhere herein.

If there is essentially no amount of brexpiprazole dimer exhibiting the characteristics as disclosed elsewhere herein, preferably exhibiting the structure of Formula I, determined, then the batch is determined as being suitable for the preparation of a pharmaceutical composition. In a preferred embodiment, the pharmaceutical composition intended to be prepared is an injectable pharmaceutical composition, such as an injectable preparation.

Finally, the present invention further refers to the use of a brexpiprazole dimer as characterized elsewhere herein, preferably characterized by the chemical structure of Formula I:

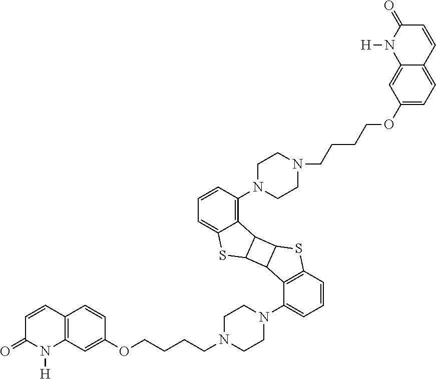 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

for evaluating the suitability of a batch of brexpiprazole dihydrate for the preparation of a pharmaceutical composition, preferably an injectable pharmaceutical composition, wherein said batch of brexpiprazole dihydrate is suitable for the preparation of a pharmaceutical composition if the amount of brexpiprazole dimer exhibiting the characteristics as disclosed elsewhere herein, preferably wherein the amount of compound of formula I, in the tested batch is at most 1.00% w/w relative to the amount of brexpiprazole dihydrate in said tested batch, more preferably it is at most 0.50%, such as at most 0.20%, for example at most 0.10%. Preferably the brexpiprazole dimer that is characterized as disclosed elsewhere herein, preferably that is characterized by the chemical structure of Formula I, is not detectable.

Other objects, features, advantages and aspects of the present invention will become apparent to those of skill from the present description and examples. It should be understood, however, that the description and the following specific examples, while indicating preferred embodiments of the invention, are given by way of illustration only. Various changes and modifications within the spirit and scope of the disclosed invention will become readily apparent to those skilled in the art from reading the description and the other parts of the present disclosure.

The present invention is further illustrated in the following examples, which are not to be construed as in any way limiting the scope of present invention.

### EXAMPLES

### Example 1—Preparation of Brexpiprazole

Crystalline brexpiprazole anhydrate was prepared according to comparative example 1 of WO 2013/162046 A1. Particle size distribution was measured with a Malvern Mastersizer 3000 laser diffraction analyzer as described elsewhere. D90 of the correspondingly measured particle size distribution of the original crystalline brexpiprazole anhydrate powder was determined to be 219 μm.

Brexpiprazole dihydrate was prepared in accordance with WO 2013/162046 A1.

### Example 2—Brexpiprazole Dihydrate is Significantly More UV-Sensitive than Brexpiprazole Anhydrate

Brexpiprazole anhydrate and brexpiprazole dihydrate were respectively exposed to UV-radiation using an appropriate illumination device (Atlas Suntester XLS+) at 250 W/m² for 60 hrs, which corresponds to a total UV light intensity of 1350 W·h/m².

The results are shown in Table 1 below.

TABLE 1

| | UV-induced Degradation |
|---|---|
| | Impurities incl. |
| Sample - Condition | Brexpiprazole Dimer |
| Anhydrate - unstressed | — |
| Anhydrate - 60 h Suntester | 0.24% |
| Dihydrate - unstressed | — |
| Dihydrate - 60 h Suntester | 3.06% |

It was observed that the dihydrate form of brexpiprazole is significantly more sensitive to UV-induced dimerization of brexpiprazole than the anhydrate form.

The UV-induced brexpiprazole dimer impurity generated from brexpiprazole dihydrate has
a UV-chromatographic RRT value of 0.92±0.02 relative to the peak for brexpiprazole, using the following HPLC conditions:

| Parameter | Dionex 3000 | | |
|---|---|---|---|
| Column | YMC Pack Pro C18 - 3.0 μm - 100 × 4.6 mm | | |
| Temperature Column | 45° C. | | |
| Compartment | | | |
| Solvent A | 0.1% acetic acid pH 2.3/ACN - 85:15 ⇒ Gradient | | |
| Solvent B | 0.1% acetic acid pH 2.3/ACN - 25:75 ⇒ Gradient | | |
| | Time [min] | % A | % B |
| *) Gradient | 0 | 100 | 0 |
| | 25 | 69 | 40 |
| | 30 | 0 | 100 |
| | 30.1 | 100 | 0 |
| | 37 | 100 | 0 |

| | |
|---|---|
| Flow | 0.8 ml/min—p ≈107 bar |
| Wavelength | 254 nm |
| Injection Volume | 10 μl |
| Temperature Auto | RMT |
| Sampler | |
| Sample Concentration | 0.5 mg/ml |
| Run Time | 37 min - RT (Brexpiprazole) ≈ 13 min |

the chemical formula C₅₀H₅₄N₆O₄S₂, and
the chemical structure of Formula I:

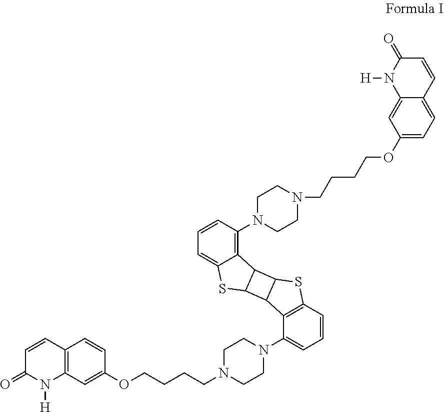 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

The UV-induced brexpiprazole dimer impurity generated from brexpiprazole anhydrate has
a UV-chromatographic RRT of 1.03±0.02 (Formula II) using the following HPLC conditions:
   HPLC: HPLC-System_Agilent_1200
   Oven temperature: 30° C.//Injection volume: 4 μl//Wavelength: 222 nm//Flow: 0.8 ml/min
   Eluent_A: H2O:H3PO4 (85%)=2000 g H2O+9.3 g H3PO4
   Eluent_B: H2O:H3PO4 (85%)/Acetonitrile=500 g H2O+9.3 g H3PO4+1183 g Acetonitrile
   Column: Symetry_C18_3.5 μm_150×4.6 mm
   Gradient: 0′ 5%; 10′ 95%; 13′ 95%; 13.5′ 5% Eluent_B
The chemical formula C₅₀H₅₄N₆O₄S₂, and
the chemical structure of Formula II:

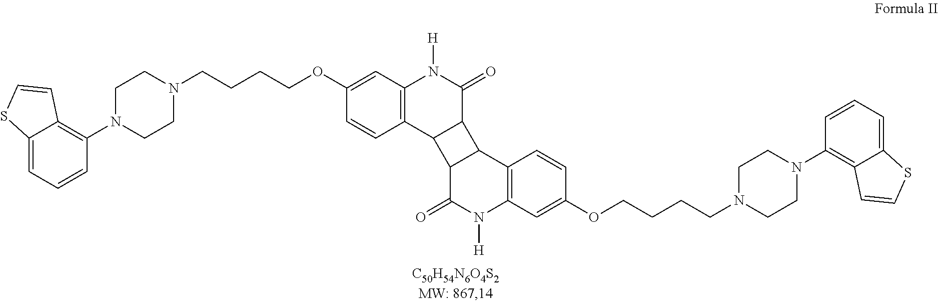 [SMILES: O=C1Nc2cc(OCCCCN3CCN(c4cccc5sccc45)CC3)ccc2C2C1C1c3ccc(OCCCCN4CCN(c5cccc6sccc56)CC4)cc3NC(=O)C21]

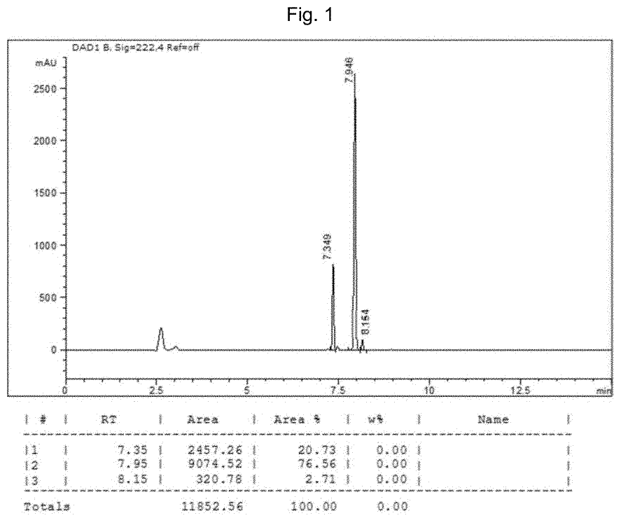

In general the compound of formula I elutes slightly earlier than brexpiprazole on a C-18 reverse phase HPLC column with an acidic water/acetonitrile mobile phase, while compound of formula II elutes slightly later than brexpiprazole, as can be seen in FIG. 1 where the compound of formula 1 elutes at 7.35 min, brexpiprazole at 7.95 min and the compound of formula II elutes at 8.15 min. Relative to brexpiprazole the RRTs (relative retention times) were 0.92 for the compound of formula I and 1.03 for the compound of formula II under the specific conditions used.

The conditions were as follows:

| Parameter | Agilent Serie 1200 | | |
|---|---|---|---|
| Column | Symetry_C18_3.5 μm_150 × 4.6 mm | | |
| Temperature Column | 35° C. | | |
| Compartment | | | |
| Solvent A | H2O + 0.1% HCOOH | | |
| Solvent B | H2O:ACNL = 8:2 + 0.1% HCOOH | | |
| | Time (min) | % A | % B |
| *) Gradient | 0 | 5 | 95 |
| | 10 | 95 | 5 |
| | 13 | 95 | 5 |
| | 13.5 | 5 | 95 |
| | 15.5 | 5 | 95 |

| | |
|---|---|
| Flow | 0.8 ml/min—p_approx. 120 bar |
| Wavelenght | 222 nm |
| Injection Volume | 4 μl |
| Temperature Auto Sampler | +4° C. |
| Sample Concentration | approx. 0.5 mg/ml |
| Run Time | 17 min |
| Retention Time Formula I | 7.35 min |
| Retention Time Brexpiprazol | 7.95 min |
| Retention Time Formula II | 8.15 min |

### Example 3—Characterization of the Compound of Formula I

The compound appearing at RRT 0.9 on the dionex 3000 column was isolated by HPLC, analyzed by HPLC-MS and H₁ NMR in DMSO-d6+FTA-d1, and compared to brexpiprazole

MS-Analysis showed that this peak was a dimer of brexpiprazole appearing with a charge z=2. H NMR analysis showed that signals characteristic of the brexpiprazole thiophen system had changed. Two signals (d, 7.53 ppm and d, 7.78 ppm) had disappeared completely, two new signals had appeared (4.63 ppm, dd, 2H and 4.44 ppm, dd, 2H), signals at 6.99 ppm, 7.34 ppm and 7.72 ppm had shifted while other olefinic signals remained essentially unaffected. The data was consistent with this dimer being compound I.

### Example 4—Characterization of the Compound of Formula II

As shown in table 1, only low quantities of brexpiprazole dimer were obtained by direct UV-irradiation of brexpiprazole anhydrate.

For the structure determination of the compound of formula II in Example 2 it was therefore necessary to produce larger quantities of that compound. Larger quantities of the compound appearing at RRT 1.03 on the Symmetry C-18 column were prepared in a 100 ml reactor, by suspending 1.0 g of brexpiprazole anhydrate in a mixture of 10.0 g toluene+10.0 g methanol at RT. The suspension was heated to 50° C. After stirring for 10 minutes, a clear solution was obtained. The solution was put into a transparent glass vial and irradiated for 18 h by a UV lamp (UVHQ 250 Z by “UV Technik Speziallampen”; radiation strength UVC/UVB/UVA: 38 W at 200 nm-280 nm; 20 W at 280 nm-315 nm; 18 W at 315 nm-400 nm). A light brown, clear solution was obtained. A mixture of brexpiprazole and compound II was obtained (19:81 area %). In a control experiment where the solution was protected from UV light by wrapping the vial in aluminum foil, brexpiprazole remained stable and compound of formula II did not appear.

The compound of formula II was purified away from brexpiprazole by chromatography on silica gel. It eluted at exactly the same position as the compound observed by UV-irradiation of crystalline brexpiprazole anhydrate. Elution was with a mixture of acetone:isopropanol (1:1 v/v). The peak representing compound of formula II was concentrated in a rotary evaporator at +50° C. at 50-30 mbar. The resulting oil was characterized by NMR, HPLC and LC-MS.

Purity by LC-MS-HPLC in area % was 93.7. Brexpiprazole in solution was significantly more sensitive to UV-radiation than brexpiprazole anhydrate.

Compound of formula II was further analyzed by HPLC-MS and H NMR (Noesy) in DMSO-d6, and compared to brexpiprazole

MS-Analysis showed that this peak was a dimer of brexpiprazole. 1H NMR analysis showed that signals characteristic of the brexpiprazole quinolone system had changed. Two signals (d, 7.83 ppm and d, 6.34 ppm) had disappeared, two new signals had appeared (d, 3.67 ppm and d, 3.45 ppm). Signals at 7.58, 6.84 and and 6.83 ppm (corresponding to the benzo part of the quinolone moiety) had shifted while other olefinic signals (corresponding to the benzothiophene moiety) remained essentially unaffected. The data was consistent with this dimer being compound II.

### Example 5—Brexpiprazole Anhydrate and Brexpiprazole Dihydrate not Only Show Different Sensitivity Towards UV-Induced Dimerization, but they Even Generate Different Dimers Upon Exposure to UV-Irradiation

Suspensions of brexpiprazole anhydrate or brexpiprazole dihydrate (about 10 mg per experiment) in different oils (about 105 mg per suspension) were prepared and irradiated for 4 hours. Analysis by HPLC (conditions as shown in example 2 for Agilent 1200) demonstrated that the different dimers compound I and compound II had formed to a very different extent depending on the respective starting material.

| | | Compound I | | |
|---|---|---|---|---|
| Solid form | Oil | (area) | Brexpiprazole | Compound II |
| Brexpiprazole | Corn oil | 0.0% | 99.8% | 0.2% |
| Anhydrate | | | | |
| Brexpiprazole | Corn oil | 32.2% | 64.9% | 3.0% |
| Dihydrate | | | | |
| Brx. Anhydrate | Peanut oil | 0.0% | 99.8% | 0.2% |
| Brx. Dihydrate | Peanut oil | 32.4% | 64.3% | 3.3% |
| Brx. Anhydrate | Castor oil | 0.0% | 99.6% | 0.4% |
| Brx. Dihydrate | Castor oil | 35.1% | 61.7% | 3.2% |

FIG. 1 shows a chromatogram of an experiment similar to the experiments with brexpiprazole dihydrate in the table above. The peaks for compound I, brexpiprazole and compound II are resolved in the chromatogram shown in FIG. 1.

The experiment showed that also in the context of theses suspensions brexpiprazole dihydrate was more sensitive to UV-induced dimerization than brexpiprazole anhydrate. Moreover, irradiation of brexpiprazole dihydrate resulted predominantly in the dimer compound of formula I, while UV-irradiation of brexpiprazole anhydrate did not produce this dimer at all, but instead gave low levels of the dimer compound II. The fact that suspensions of brexpiprazole dihydrate also yielded small amounts of the dimer compound of formula II might be explained by dimerization of the small amount of brexpiprazole dissolved in the oil phase of the suspension, because it is known from example 4 that dissolved brexpiprazole can yield compound of formula II upon UV-irradiation.

Without wishing to be bound to any theory, the data appears consistent with the following model:

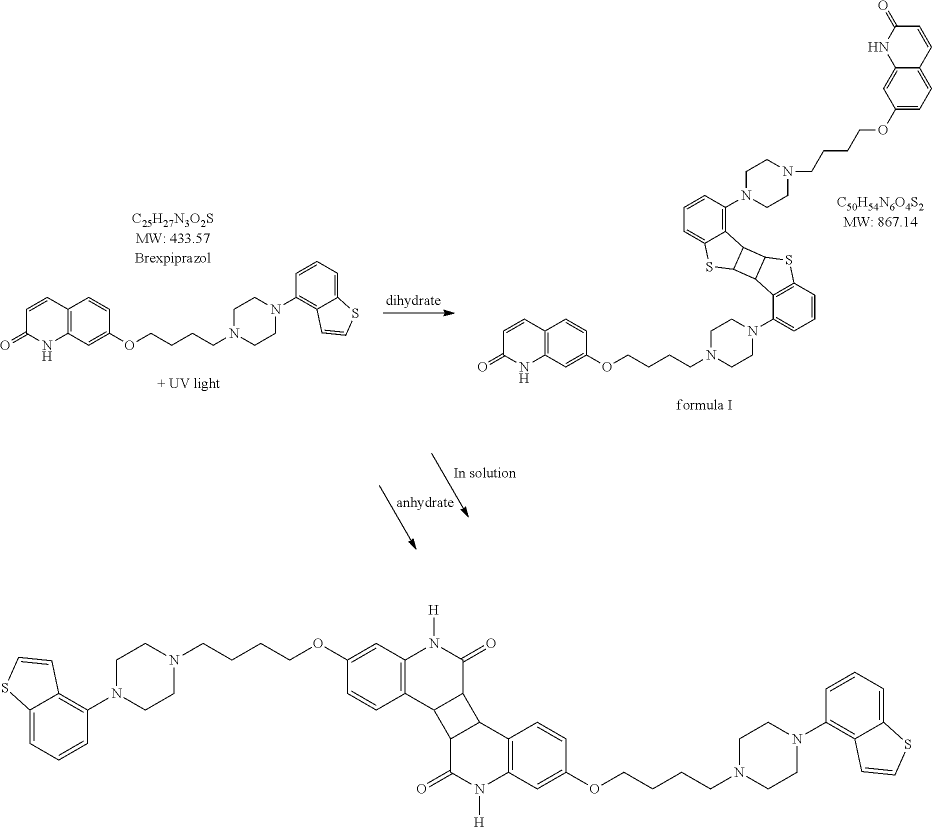 [SMILES: O=c1ccc2ccc(OCCCCN3CCN(c4cccc5sccc45)CC3)cc2[nH]1 ; O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1 ; O=C1Nc2cc(OCCCCN3CCN(c4cccc5sccc45)CC3)ccc2C2C1C1c3ccc(OCCCCN4CCN(c5cccc6sccc56)CC4)cc3NC(=O)C21]

1-2. (canceled)

3. A package including one or more pharmaceutical compositions comprising brexpiprazole dihydrate, wherein said package comprises a packaging material, which at least partially encloses said one or more pharmaceutical compositions and which is capable of blocking, absorbing and/or reflecting UV exposure up to a wavelength of 450 nm.

4. The package according to claim 3, wherein the packaging material comprises aluminum foil and/or polyvinyl chloride (PVC) or polyvinylidene chloride (PVDC); or glass, respectively selected to block, absorb and/or reflect UV exposure up to a wavelength of 450 nm.

5. A process for producing a pharmaceutical composition comprising brexpiprazole dihydrate, wherein during manufacturing a composition comprising brexpiprazole dihydrate is protected against exposure to UV irradiation up to a wavelength of 450 nm, in order to reduce or prevent UV-induced brexpiprazole dimerization during production.

6. A manufacturing system adapted for carrying out a process according to claim 5.

7. A pharmaceutical composition comprising brexpiprazole dihydrate, wherein the amount of brexpiprazole dimer is at most 1.00% w/w relative to the amount of brexpiprazole dihydrate, and wherein the brexpiprazole dimer is characterized by anyone of the following characteristics (i) to (iv), respectively alone or in combination:
(i) UV-chromatographic RRT of 0.92±0.02 (Formula I)
(ii) LC-MS molecular weight [M+H]+=867±1,
(iii) chemical formula C₅₀H₅₄N₆O₄S₂, or
(iv) Chemical structure of Formula I

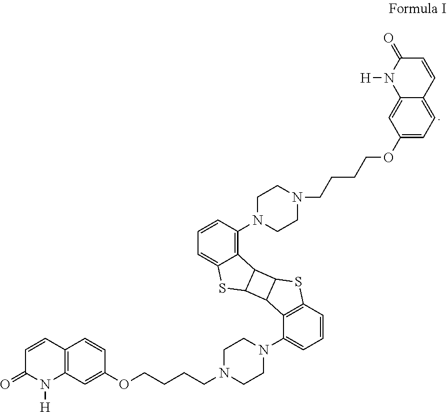 [SMILES: O=c1ccc2ccc(OCCCCN3CCN(c4cccc5c4C4C(S5)C5c6c(cccc6N6CCN(CCCCOc7ccc8ccc(=O)[nH]c8c7)CC6)SC45)CC3)cc2[nH]1]

8. The pharmaceutical composition according to claim 7, being a suspension.

9. Injectable pharmaceutical preparation, comprising the suspension according to claim 8.

10. Vial or prefilled syringe comprising the pharmaceutical composition according to claim 7.

11. The vial or prefilled syringe according to claim 10, wherein the vial or syringe comprises material capable to block, absorb and/or reflect UV exposure in a wavelength region of up to 450 nm.

12. The vial or prefilled syringe according to claim 11, wherein the material capable to block, absorb and/or reflect UV exposure is borosilicate glass.

13. A method of evaluating the suitability of a batch of brexpiprazole dihydrate for the preparation of a pharmaceutical composition, comprising the steps of:
(1) providing a sample of the batch of brexpiprazole dihydrate;
(2) determining the presence or absence of a brexpiprazole dimer that is characterized as defined in claim 7,
(3) determining that the batch is suitable for the preparation of a pharmaceutical composition if the amount of the brexpiprazole dimer as determined in (2) is at most 1.0% w/w relative to the amount of brexpiprazole dihydrate.

14. (canceled)

15. The pharmaceutical composition as defined in claim 7, which provides a unit dose, for the treatment of a central nervous system disease.

16. A method for reducing or preventing UV-induced dimerization of brexpiprazole in a brexpiprazole dihydrate comprising pharmaceutical composition, the method comprising:
at least partially enclosing a pharmaceutical composition comprising brexpiprazole dihydrate in a substance or a material which is capable of blocking, absorbing and/or reflecting UV exposure up to a wavelength of 450 nm.
